(19) United States
(12) Patent Application Publication
(10) Pub. No.: US 2005/0142083 A1
(43) Pub. Date: Jun. 30, 2005

(54) Natural pearlescent odor reduction

(76) Inventors: Leila Song, Putnam Valley, NY (US); Dennis F. Turner, Eastport, ME (US); Robert A. Lewis, Eastport, ME (US)

Correspondence Address: The Law Office of Stuart D. Frenkel, P.C., Suite 330, 3975 University Drive, Fairfax, VA, 22030, US

(21) Appl. No.: 10/747,668

(22) Filed: Dec. 29, 2003

(51) Int. Cl. A61K 7/021; A61K 7/00

(52) U.S. Cl. 424/63; 424/76.1

(57) ABSTRACT

Natural pearlescent pigment derived from fish is deodorized by contacting the pigment with a complex metal hydride. Addition of a weak acid subsequent to treatment with the complex metal hydride enhances the deodorization process.

### FIELD OF THE INVENTION

The present invention is directed to improved natural pearlescent pigments and, in particular, to a process for reducing the odor of natural pearlescent pigments.

### BACKGROUND OF THE INVENTION

Laminar or plate-like pigments which impart a pearly or nacreous luster into objects on which or in which they are used are known as “effect” pigments, and have also been known as pearlescent pigments or nacreous pigments. These effect pigments include naturally occurring substances such as pearlescence, a mixture of guanine and hypoxanthine that is obtained from fish.

The manufacture of pearl essence from natural products was primarily a European industry until the First World War when the United States became a significant manufacturer of this product.

When fish die, the by-products formed during the process of decay are very destructive to the facets of the small organic guanine crystals. Accordingly, the faster the crystals are isolated from the fish, the better is the quality of the pearl essence which is obtained. To expedite the process, only those fish which very easily shed their scales are used and in America, fish of the herring family have been found to be the most suitable. These fish are gathered up by the hundreds of thousands in a net, transferred to the hold of a boat and as the fish struggle for room and “breathing space,” they rub against each other and this causes the scales to detach. The scales are collected and the guanine crystals are isolated therefrom.

In broad terms, the scales were originally agitated or scrubbed with water, which may be warm, or which may have contained ammonia or chemicals which acted as washing compounds. The crystals were isolated and washed. Thereafter, the crystals were thinned with ammonia water and mixed with a lacquer which has a greater affinity for the crystals. The lacquer was isolated and used as a pearlescent paste.

For additional background, one may consult Mattin, Pearl Essence Facts, page 13 (September 1932) and a pamphlet published by Rinshed-Mason Company entitled “Pearl Essence, Historical and Descriptive Data.”

It is a common practice that toiletries, such as shampoos, hair rinses, lotions, creams, soaps, cosmetics, and the like are imparted with pearlescence in order to improve their attractiveness and to enhance their value as commercial products. Hitherto known pearlescent agents used to impart such pearlescence are thin leaf materials of natural origin such as natural crystalline guanine and mica, of which the former is particularly preferred.

Natural crystallized guanine, however, may contain impurities, believed to be amines, which can cause deterioration of and as well provide an unpleasant odor in the products formulated therewith. The unpleasant odor may still persist even after several bleaching and washing steps. Accordingly, as a recent trend in the industry of toiletries, natural crystallized guanine is being replaced with synthetic materials which are more readily available and capable of exhibiting pearlescence almost as good as that obtained with the pearlescent agents made from fish. The effect pigments which are most often encountered commercially are titanium dioxide-coated mica and iron oxide-coated mica. Other synthetic effect pigments which have been developed for both cosmetic and industrial use include materials such as bismuth oxychloride and lead carbonate.

In addition to the high cost and limited supply, and an inability to achieve a high solids content without destroying crystalline structure, natural pearl pigments have limited industrial applications. However, natural pearl essence has a satiny luster that creates soft, cloud-like mists and deep luster. Many cosmetic and personal care products contain natural pearl pigments to increase luster, depth, iridescence, and pearlescence, and to provide for a soft, shimmering, pearly effect product.

Accordingly, the need for natural pearlescent pigments still remains for generating quality color effects in a variety of commercial applications including, for example, cosmetics. It is important, however, that the natural pearlescent agents be odor free.

U.S. Pat. No. 4,966,734 discloses a process for removing undesirable odors from fatty ester mixtures which, in addition to other components, contain the esters of highly unsaturated fatty acids, in particular those of eicosapentaenoic acid (EPA) and docosahexaenoic acid (DHA) derived from fish oils. One problem in the preparation of EPA- and DHA-containing preparations from fish oils for oral consumption is the fish odor, which is extremely stubborn and remains even after transesterification and concentration of EPA and DHA. This odor is due to a large number of compounds which are formed by oxidative degradation of highly unsaturated fatty acids (cf. J. Amer. Oil Chem. Soc. 52 (1975), 349-353). These are predominantly unsaturated carbonyl compounds, which can have an intense odor even at very low concentrations. Deodorization is carried out in a simple manner by thoroughly stirring the ester mixture with a solution of a complex hydride, such as an aqueous sodium borohydride solution.

### SUMMARY OF THE INVENTION

It has been found, surprisingly, that deodorization of natural pigments from fish can be carried out in a simple manner by contacting the natural crystallized guanine with a complex hydride, such as a sodium borohydride. Deodorization is achieved without significant crystal degradation and associated loss of luster caused by crystal fragmentation. In this procedure, it is believed the compounds that cause the fish odor are removed from the pearlescent material or otherwise reduced or neutralized.

### DETAILED DESCRIPTION OF THE INVENTION

The present invention therefore relates to a process for removing undesirable odors from a mixture of guanine and hyperxanthine crystals obtained from fish by treating the mixture with a complex hydride. The process is particularly important for removing undesirable odors from natural pearlescent pigments obtained from fish for use in cosmetic or other applications that benefit from the color effect derived from such pigments.

According to the invention, the complex metal hydrides that can be used for deodorization are compounds such as lithium aluminum hydride, sodium borohydride and lithium borohydride. NaBH₄ is particularly advantageously used. The complex hydride is contacted with the pearlescent pigment which is in the form of a paste. Subsequent neutralization with a weak acid completes the deodorization process.

The initial steps of the process involve separating the natural pearlescent pigment from the fish and forming a pearlescent paste. These steps are known in the art and do not, per se, form part of the novelty of this invention. The process set forth below represents one way for initially washing and/or treating the crystals to form a paste that can be treated according to the present invention to reduce odor. Any other method which can yield a clean crystal ready for incorporation into products such as cosmetics can be used prior to the deodorization treatment.

The first step involves separating the native guanine crystals from the fish such as the scales. This step employs a hot water washing. It has been found that if the water is too cold, only a small portion of the crystals will be removed from the scales and if the water is too hot, it will cause the scales to curl thereby making the crystals largely inaccessible for extraction. It has been found that the water temperature should be about 30° C. to 50° C. and preferably from about 34° C. to 40° C.

A quantity of water sufficient to extract the guanine is combined with the fish scales which may have been previously washed with cold or warm (20° C.) water to remove extraneous matter. Conveniently, the amount of water is in the range of about 1 ½ to 2 ½ times the weight of the scales which, in general, have a total solids content of about 30% to 40%. Under these circumstances, adding water having a temperature of about 50° C. results in a final mixing temperature of about 34° C. to 40° C., the range of optimum extraction of the pearl essence crystals.

The mixing of the scales with the hot water is accomplished with agitation. This may be affected using various mixing devices such as troughs with ribbon screws or tanks with propeller agitators. Another alternative is to use a low shear pump such as that used for transferring fruit and vegetables. The duration of the agitated mixing will vary depending on the condition of the scales being treated but, in general, ranges from about 5 to 15 minutes.

The resulting extract liquor is separated from the extracted scales by any suitable means such as a screen or filter. If desired, the separated scales can be rinsed with water, one or more times, at temperatures ranging from ambient to about 50° C. It is preferred to combine and rinse liquor, after separation from the scales, with the initial extract liquor.

The aqueous extract is then concentrated by, for example, gravity settling, centrifugation, or combinations thereof. Using centrifugation, the pearl essence crystals are recovered in the form of an aqueous paste. In one preferred procedure, the combined extract and rinse liquids are permitted to settle for an extended period of time, e.g., overnight, during which time the major amount of the pearl essence crystals accumulate in the lower fraction, which generally comprises 15% to 25% of the total volume. This lower fraction is separated providing a concentrated aqueous slurry. The top portion of the settled extract/rinse water can be centrifuged to recover any pearl essence crystals which may be present and the resulting water can be reused in the first step of the present process. The product is a concentrated aqueous pearl essence slurry or paste which contains a significant quantity of impurities. It is therefore subjected to second stage processing.

In the second stage, pearl essence crystals are preferentially transferred from the concentrated aqueous paste into the organic phase by the method of flushing. As a result of the transfer, most of the extraneous material is separated from the crude pearl essence concentrate. Organic flushing agents which can be employed in the process of the present invention include aliphatic and aromatic hydrocarbons, castor oil, soybean oil, jojoba oil, mineral oil, naphtha, isoparaffins, lanolin oil, lard oil, lecithin, organic esters of long chain alcohols such as octyl acetate, and various other vegetable and fish oils as well as mixtures of organic liquids and surfactants. Preferably, the organic contains about 0.1 to 10 wt %, preferably about 4 to 6 wt %, of a nonionic surfactant such as polyoxyethylene sorbitan monooleate, or an alkyl sulfosuccinate such as Aerosol OT, or a fatty acid salt such as sodium oleate, and the like. The organic flushing agent will generally comprise about 30% to 60% by weight based on the weight of the resultant flushed paste. The combination of the organic flushing agent and concentrated extract is mixed and sufficient mixing can be determined by observation. Thus, the mixing is deemed to be at an appropriate level when the pearl crystals combine with the organic solvent to form small beads which can be separated from the water phase which contains most of the impurities found in the original concentrate. In general, the mixing is continued for about ½ of an hour to one hour.

After separating the flushed pearl essence paste from the water by any suitable means, such as a screen or filter, the flushed paste can be further washed with water, ammonia water, or water containing a small amount of surfactant. The resulting product of the invention is free from most impurities, has good storage characteristics, and is ready to be refined into final form by a process which is dependent on the type of product desired.

Either immediately from the washing step or from storage, the pearlescent pigment paste, which comprises approximately 30-40% of the pearlescent pigment material, is treated with the complex hydride. As sodium borohydride (NaBH₄) is the preferred complex hydride, the process will be explained with the use of such material. It is to be understood equivalent complex hydrides, including those previously described can be used in place of the sodium borohydride compound. The sodium borohydride can be applied to the pearlescent pigment paste in aqueous solution in concentrations preferably above 10 wt %. However, to avoid immediate and excessive hydrogen off-gassing, the sodium borohydride is preferably added to the pigment paste as a powder. The sodium borohydride powder, for example, can be sprinkled or otherwise applied onto the paste. The amount of the sodium borohydride applied whether in the form of an aqueous solution or solid will range from about 0.5 to about 10% by weight sodium borohydride relative to the pigment paste. More typical amounts of the sodium borohydride added to the pigment paste range from about 0.75-5 wt. % and, more preferably, about 1 wt. % of the sodium borohydride relative to the pearlescent paste is added. The pearlescent pigment paste and sodium borohydride powder are mixed until a uniform mixture is achieved. Any known type of mixing equipment can be used. The pearlescent pigment paste and sodium borohydride are mixed for about two minutes to two hours, more typically for less than one hour and more preferably from about three to ten minutes to form a uniform mixture.

Temperature of treatment and mixing will generally be at ambient conditions. Temperatures up to about 50° C. can be utilized. Upon forming a uniform mixture, sufficient water is added to form a flowable or pumpable paste. Some water may be added to enhance mixing. Typically the amount of water added to the pearlescent pigment paste will range from about 50% to about 200%. More typically, about 100% by weight water relative to the pigment paste is needed to form a flowable or pumpable mixture.

Subsequent to the formation of a pumpable liquid dispersion of pearlescent pigment paste, sodium borohydride, and water, a small amount ranging from about 0.5 to 10 wt. %, preferably about 0.75 to 5 wt %, and, more preferably, about 1 wt %, of a weak acid relative to pearlescent paste is added to the pumpable mixture or slurry. The addition of the weak acid neutralizes both the finished product and the liquid wastewater, providing improved odor reduction. Again, ambient temperature conditions up to 50° C can be utilized. The process is characterized by foaming and off-gassing from the slurry. This foaming and off-gassing of hydrogen will happen when mixing just the water, sodium borohydride and acetic acid without the pearl paste. It is important to add the weak acid soon after the mixture of pearlescent pigment paste, sodium borohydride, and water are provided in a pumpable slurry. Delaying the acid neutralization prevents or reduces the significant off-gassing which appears to be needed to provide successful odor reduction. Accordingly, if the mixture is allowed to sit too long, the addition of the acid does not result in the desired off-gassing or foaming action which is needed. Accordingly, delays of adding the acid once the mixture of pearlescent pigment paste and sodium borohydride is provided should not generally exceed three hours. Once the off-gassing and foaming subsides, additional water can be added to rinse away residual materials. The slurry can then be separated such as by centrifugal action and the pearlescent pigment material which is separated can be dispersed into the appropriate vehicle in which it will eventually be utilized. It has been found that a high concentration of pearlescent pigment paste for a given time of centrifugation at a given G strength is achieved. Accordingly, the centrifugation time can be slightly reduced to yield the desired product. The treated pearlescent pigment product easily disperses into both aqueous solutions and organic solvent dispersions using typical agents that are generally accepted by current practice as known by those of ordinary skill in the art.

While not wishing to be bound by any particular theory, it is believed that the odor contamination in the pearlescent pigment material derived from fish is influenced by amine compounds. The borohydride-acid system which is used to treat the pearlescent pigment paste is thought to affect the odor diminution through a first reduction of odoriferous compounds including lower amines such as dimethyl amine and a second weak Lewis acid-base reaction to release the reduced compounds.

The weak acids which can be added to the slurry of pearlescent pigment paste and borohydride to induce the off-gassing and foaming of the borohydride-treated pearlescent pigment paste are those with relatively low disassociation constants, unlike strong acids such as sulfuric or phosphoric acids. A non-limiting list of useful weak acids includes organic acids such as formic acid, acetic acid, C₃₊ alkanoic acids, citric acid, malic acid, lactic acid, etc. Weak inorganic acids such as nitric and nitrous acid can also be used. Acetic acid is preferred.

The deodorized color effect materials of the invention are advantageous for many purposes, such as the coloring of paints, printing inks, plastics, glasses, ceramic, and decorative cosmetic preparations.

Products of this invention have use in all types of automotive paint applications. For example, these effect pigments can be used in mass tone or as styling agents to spray paint all types of automotive and non-automotive vehicles. Similarly, they can be used on all clay/formica/wood/glass/metal/enamel/ceramic and non-porous or porous surfaces. The effect pigments can be used in coating compositions or incorporated into plastic articles geared for the toy industry or the home. These effect pigments can be impregnated into fibers to impart new and esthetic coloring to clothes and carpeting. They can be used to improve the look of shoes, rubber and vinyl/marble flooring, vinyl siding, and all other vinyl products. In addition, these colors can be used in all types of modeling hobbies. Natural Pearl Pigments have limited industrial applications, again due to temperature, pH, shear, cost and an inability to achieve high total solids content without destroying crystalline structure. The natural pigment total solids range is typically kept below 40% and typically sold at around 20%.

The above-mentioned compositions in which the compositions of this invention are useful are well known to those of ordinary skill in the art. Examples include printing inks, nail enamels, lacquers, thermoplastic and thermosetting materials, natural resins, and synthetic resins. Some non-limiting examples include polystyrene and its mixed polymers, polyolefins, in particular, polyethylene and polypropylene, polyacrylic compounds, polyvinyl compounds, for example polyvinyl chloride and polyvinyl acetate, polyesters and rubber, and also filaments made of viscose and cellulose ethers, cellulose esters, polyamides, polyurethanes, polyesters, for example polyglycol terephthalates, and polyacrylonitrile.

For a well-rounded introduction to a variety of pigment applications, see Temple C. Patton, editor, The Pigment Handbook, volume 11, Applications and Markets, John Wily and Sons, New York (1973). In addition, see for example, with regard to ink: R. H. Leach, editor, The Printing Ink Manual, Fourth Edition, Van Nostrand Reinhold (International) Co. Ltd., London (1988), particularly pages 282-591; with regard to paints: C.H. Hare, Protective Coatings, Technology Publishing Co., Pittsburgh (1994), particularly pages 63-288. The foregoing references are hereby incorporated by reference herein for their teachings of ink, paint, and plastic compositions, formulations and vehicles in which the compositions of this invention may be used including amounts of colorants.

In the cosmetic field, the effect materials can be used in all cosmetic and personal care applications subject, of course, to all regulatory requirements. Thus, they can be used in hair sprays, leg-makeup, insect repellant lotion, mascara cake/cream, nail enamel, nail enamel remover, perfume lotion, and shampoos of all types (gel or liquid). In addition, they can be used in shaving cream (concentrate for aerosol, brushless, lathering), skin glosser stick, skin makeup, hair groom, eye shadow (liquid, pomade, stick, pressed, or cream), eye liner, cologne stick, cologne, cologne emollient, bubble bath, body lotion (moisturizing, cleansing, analgesic, astringent), after shave lotion, after bath milk, and sunscreen lotion.

For a review of cosmetic applications, see Cosmetics: Science and Technology, 2^{nd} Ed., Eds: M.S. Balsam and Edward Sagarin, Wiley-Interscience (1972) and deNavarre, The Chemistry and Science of Cosmetics, 2^{nd} Ed., Vols 1 and 2 (1962), Van Nostrand Co Inc., Vols 3 and 4 (1975), Continental Press, both of which are hereby incorporated by reference.

### EXAMPLE 1

The following Table sets forth differing treatments of the pearlescent paste in order to compare the processes for odor reduction. It can be seen Sample 2 provided the best odor reduction. Odor of the samples were evaluated by a subjective human smell test.

TABLE 1
ODOR EVALUATIONS¹

| | | | | | | | | | | |
|---|---|---|---|---|---|---|---|---|---|---|
| SAM- | PEARL | PASTE | MTC LAC #87 | MIX | WATER | ACETIC | WATER | | CENTRIFUGED | FISH |
| PLE 1 | ANALYSIS | WEIGHT | (3% Methocel | Mix well | ADDED | ACID 0 | ADDED 0 | | 20 min | ODOR |
| | 32% | 10 | 97%) (water) | | 50 | | | | @ 1800 rpm | Live Fish |
| SAM- | PEARL | PASTE | SODIUM² | MIX | WATER | ACETIC ACID | WATER | | CENTRIFUGED | FISH |
| PLE 2 | ANALYSIS | WEIGHT | BORO- | 5 minutes | ADDED | 0.1 mix 5 min | ADDED | | 30 min | ODOR |
| | 32% | 10 | HYDRIDE 0.1 | | 10 | | 40 | | @ 1800 rpm | None |
| SAM- | PEARL | PASTE | MTC LAC #87 | MIX | WATER | SODIUM² | ACETIC | WATER | CENTRIFUGED | FISH |
| PLE 3 | ANALYSIS | WEIGHT | (3% Methocel | Mix well | ADDED | BORO- | ACID | ADDED | 20 min | ODOR |
| | 32% | 10 | 97%) (water) | | 10 | HYDRIDE 0.1 | 0.1 | 40 | @ 1800 rpm | Some |
| | | | | | grams | | | | | |
| | | | | | mix well | | | | | |
| SAM- | PEARL | PASTE | MTC LAC #87 | MIX RAW | WATER | SODIUM | ACETIC | WATER | CENTRIFUGED | FISH |
| PLE 4 | ANALYSIS | WEIGHT | (3% Methocel | CONTROL | ADDED 0 | BORO- | ACID 0 | ADDED 0 | 0 | ODOR |
| | 32% | 10 | 97%) (water) | | | HYDRIDE 0 | | | | Very |
| | | | NO ADDS | | | | | | | Heavy |

¹All amounts are by grams unless otherwise indicated.
²Added as solid.

1. A process for the deodorization of pigment derived from fish comprising contacting said pigment with a complex metal hydride.

2. The process of claim 1 wherein said pigment is a mixture of guanine and hypoxanthine.

3. The process of claim 1 wherein said pigment is in the form of a paste comprising pigment and water when contacted with said complex metal hydride.

4. The process of claim 1 wherein said complex metal hydride is sodium borohydride.

5. The process of claim 1 comprising mixing said complex metal hydride and said pigment to form a mixture and subsequently adding a weak acid to cause foaming and off-gassing from said mixture.

6. The method of claim 5 wherein said complex metal hydride is sodium borohydride.

7. The process of claim 6 wherein said weak acid is an organic acid.

8. The process of claim 7 wherein said organic acid is acetic acid.

9. The process of claim 6 wherein said pigment is in the form of a paste comprising pigment and water, mixing said paste with sodium borohydride to form a mixture, said sodium borohydride being added to said mixture in amounts ranging from 0.5-10 wt % of said pigment paste.

10. The process of claim 9 wherein said sodium borohydride is added as a solid powder.

11. The process of claim 10 wherein said sodium borohydride powder is added in amounts of 1 wt % relative to said pigment paste.

12. The process of claim 9 wherein said weak acid is added in amounts of from about 0.5 to 10 wt % of said pigment paste.

13. The process of claim 12 wherein said weak acid is added in a mounts of about 1 wt % relative to said pigment paste.

14. The process of claim 9 wherein said weak acid is added to said mixture of pigment paste and sodium borohydride no more than three hours after said mixture is formed.

15. The process of claim 14 wherein said weak acid is added to said mixture of pigment paste and sodium borohydride less than one hour after said mixture is formed.

16. The process of claim 9 wherein said weak acid is an organic acid.

17. The process of claim 16 wherein said organic acid is acetic acid.

18. A cosmetic formulation containing the pigment treated in accordance with the process of claim 1.

19. A cosmetic formulation containing the pigment treated in accordance with the process of claim 5.

20. A cosmetic formulation containing the pigment treated in accordance with the process of claim 10.
